(19) United States
(12) Patent Application Publication
(10) Pub. No.: US 2013/0131415 A1
(43) Pub. Date: May 23, 2013

(54) Process for Ethylbenzene Production From Ethanol

(71) Applicants: James R. Butler, Spicewood, TX (US); Aspen Texada, Houston, TX (US); William Sheets, Kingwood, TX (US)

(72) Inventors: James R. Butler, Spicewood, TX (US); Aspen Texada, Houston, TX (US); William Sheets, Kingwood, TX (US)

(73) Assignee: FINA TECHNOLOGY, INC., Houston, TX (US)

(21) Appl. No.: 13/302,526

(22) Filed: Nov. 22, 2011

(51) Int. Cl. C07C 2/66

(52) U.S. Cl. 585/323; 585/454; 585/449

(57) ABSTRACT

A method of producing an alkylaromatic by the alkylation of an aromatic with ethanol, such as producing ethylbenzene by an alkylation reaction of benzene, is disclosed.

### FIELD

Embodiments of the present disclosure generally relate to the production of ethylbenzene.

### BACKGROUND

Alkylation reactions generally involve contacting a first aromatic compound with an alkylation agent in the presence of a catalyst to form a second aromatic compound. One important alkylation reaction is the reaction of benzene with ethylene in the production of ethylbenzene. The ethylbenzene can then be dehydrogenated to form styrene.

One potential issue in the production of ethylbenzene is the availability, cost, and desirability of the components used to manufacture ethylbenzene. For instance, the use of ethylene as an alkylation source can be problematic, as ethylene is traditionally manufactured by the dehydrogenation of natural gas components. As such, it is a non-bio-sourced raw material.

In view of the above, it would be desirable to have an effective method to produce ethylbenzene in commercial quantities from a bio-sourced raw material. It would further be desirable if the method was robust and did not experience frequent disruptions due to process interruptions for catalyst regeneration or replacement.

### SUMMARY

Embodiments of the present disclosure include a method of producing ethylebenzene by the catalytic alkylation of benzene with ethanol.

In one embodiment of the present disclosure, a method of producing an alkylaromatic is disclosed which includes contacting an aromatic with ethanol in the presence of a catalyst at liquid phase alkylation conditions to form the alkylaromatic.

In another embodiment of the present disclosure, a method of producing an alkylaromatic is disclosed. The method includes providing at least one reaction zone containing a zeolite catalyst, introducing a feed stream comprising an aromatic and ethanol to the reaction zone, and, reacting at least a portion of the aromatic under alkylation conditions to produce an alkylaromatic.

In still another embodiment of the present invention, a process for producing an alkylaromatic compound is disclosed. The process includes introducing an input stream comprising an aromatic hydrocarbon, and an alkylating agent comprising ethanol into a preliminary alkylation system. The preliminary alkylation system includes a preliminary alkylation catalyst having a first SiO₂/Al₂O₃ ratio. The preliminary alkylation catalyst is a molecular sieve. The method further includes operating the preliminary alkylation system under alkylation conditions to produce the alkylaromatic compound and withdrawing from the preliminary alkylation system a first output stream. The first output stream includes the alkylaromatic compound and unreacted aromatic hydrocarbon. The process further includes introducing at least part of the first output stream and ethanol into a first alkylation system. The first alkylation system includes a first alkylation catalyst having a second SiO₂/Al₂O₃ ratio. The first alkylation catalyst is a molecular sieve, wherein the preliminary alkylation catalyst and the first alkylation catalyst are different in that the preliminary alkylation catalyst has a lower SiO₂/Al₂O₃ ratio than the first alkylation catalyst. The frequency at which any alkylation catalyst is removed for replacement, regeneration or reactivation is reduced as compared to either alkylation catalyst alone. The process also includes operating the first alkylation system under alkylation conditions to produce the alkylaromatic compound and withdrawing from the first alkylation system a second output stream including the alkylaromatic compound.

In yet another embodiment of the present disclosure, a process of producing ethylbenzene by the alkylation of benzene with ethanol is disclosed. The process includes providing at least one reaction zone comprising a zeolite catalyst, introducing a feed stream comprising benzene and ethanol to the reaction zone, and reacting at least a portion of the benzene with ethanol under alkylation conditions to produce ethylbenzene.

### BRIEF DESCRIPTION OF DRAWINGS

FIG. 1 is a schematic block diagram of an embodiment of an alkylation/transalkylation process.

FIG. 2 is a schematic block diagram of an embodiment of an alkylation/transalkylation process that includes a preliminary alkylation step.

FIG. 3 is a schematic illustration of a parallel reactor system that can be used for a preliminary alkylation step.

FIG. 4 illustrates one embodiment of an alkylation reactor with a plurality of catalyst beds.

FIG. 5 is a graphical depiction of the benzene to ethanol molar feed ratio as described for the liquid-phase reaction in Example 1.

FIG. 6 is a graphical depiction of the ethylbenzene content in the reactor effluent as described for the liquid-phase reaction in Example 1.

FIG. 7 is a graphical depiction of the benzene to ethanol molar feed ratio as described for the gas-phase reaction in Example 2.

FIG. 8 is a graphical depiction of the ethylbenzene content in the reactor effluent as described for the gas-phase reaction in Example 2.

### DETAILED DESCRIPTION

A detailed description will now be provided. Each of the appended claims defines a separate invention, which for infringement purposes is recognized as including equivalents to the various elements or limitations specified in the claims.

Various terms as used herein are shown below. To the extent a term used in a claim is not defined below, it should be given the broadest definition persons in the pertinent art have given that term as reflected in printed publications and issued patents at the time of filing. Further, unless otherwise specified, all compounds described herein may be substituted or unsubstituted and the listing of compounds includes derivatives thereof.

The term “activity” refers to the weight of product produced per weight of the catalyst used in a process per hour of reaction at a standard set of conditions (e.g., grams product/gram catalyst/hr).

The term “alkylation” refers to the addition of an alkyl group to another molecule.

The term “deactivated catalyst” refers to a catalyst that has lost enough catalyst activity to no longer be efficient in a specified process. Such efficiency is determined by individual process parameters. Further, the time from introduction of the catalyst to a system to the point that the catalyst is a deactivated catalyst is generally referred to as the catalyst life.

The term “processing” is not limiting and includes agitating, mixing, milling, blending and combinations thereof, all of which are used interchangeably herein. Unless otherwise specified, the processing may occur in one or more vessels, such vessels being known to one skilled in the art.

The term “recycle” refers to returning an output of a system as input to either that same system or another system within a process. The output may be recycled to the system in any manner known to one skilled in the art, for example, by combining the output with an input stream or by directly feeding the output into the system. In addition, multiple input/recycle streams may be fed to a system in any manner known to one skilled in the art.

The term “regeneration” refers to a process for renewing catalyst activity and/or making a catalyst reusable after its activity has reached an unacceptable/inefficient level. Examples of such regeneration may include passing steam over a catalyst bed or burning off carbon residue, for example.

The term “molecular sieve” refers to a material having a fixed, open-network structure, usually crystalline, that may be used to separate hydrocarbons or other mixtures by selective occlusion of one or more of the constituents, or may be used as a catalyst in a catalytic conversion process. The term “zeolite” refers to a molecular sieve containing a silicate lattice, usually in association with some aluminum, boron, gallium, iron, and/or titanium, for example. In the following discussion and throughout this disclosure, the terms molecular sieve and zeolite will be used more or less interchangeably. One skilled in the art will recognize that the teachings relating to zeolites are also applicable to the more general class of materials called molecular sieves.

Further, various ranges and/or numerical limitations may be expressly stated below. It should be recognized that unless stated otherwise, it is intended that endpoints are to be interchangeable. Further, any ranges include iterative ranges of like magnitude falling within the expressly stated ranges or limitations.

Embodiments of the present disclosure generally relate an alkylation system adapted to minimize process upsets due to alkylation catalyst deactivation and the resulting catalyst regeneration or replacement using an aromatic compound together with ethanol as an alkylation source. In certain embodiments of the disclosure, a large-pore catalyst is used within a liquid-phase alkylation process to produce ethylbenzene from benzene and ethanol. The process can include one or more fixed catalyst beds of large-pore catalysts that can be regenerated either in-situ or ex-situ without significant disruptions to the commercial alkylation production rates.

In certain embodiments of the liquid phase alkylation, the alkylation catalyst is a zeolite catalyst. Such catalysts include zeolite beta, zeolite Y, zeolite MCM-22, zeolite MCM-36, zeolite MCM-49 or zeolite MCM-56, for example. In one specific embodiment, the alkylation catalyst is Zeolyst CP 787 H-Beta Extrudate, available from Zeolyst International. In one embodiment, the catalyst is a zeolite beta having a silica to alumina molar ratio (expressed as SiO₂/Al₂O₃ ratio) of from about 5 to about 200 or from about 20 to about 100, for example. In one embodiment, the zeolite beta may have a low sodium content, e.g., less than about 0.2 wt. % expressed as Na₂O, or less than about 0.02 wt. %, for example. The sodium content may be reduced by any method known to one skilled in the art, such as through ion exchange, for example. (See, U.S. Pat. No. 3,308,069 and U.S. Pat. No. 4,642,226 (formation of zeolite beta), U.S. Pat. No. 4,185,040 (formation of zeolite Y), U.S. Pat. No. 4,992,606 (formation of MCM-22), U.S. Pat. No. 5,258,565 (formation of MCM-36), WO 94/29245 (formation of MCM-49) and U.S. Pat. No. 5,453,554 (formation of MCM-56), which are incorporated by reference herein.)

In one specific embodiment, the alkylation catalyst includes a rare earth modified catalyst, such as a cerium, lanthanum, praseodymium, or ytterbium promoted zeolite catalyst. In one embodiment, the cerium promoted zeolite catalyst is a cerium promoted zeolite beta catalyst. The cerium promoted zeolite beta (e.g., cerium beta) catalyst may be formed from any zeolite catalyst known to one skilled in the art. For example, the cerium beta catalyst may include zeolite beta modified by the inclusion of cerium. Any method of modifying the zeolite beta catalyst with cerium may be used. For example, in one embodiment, the zeolite beta may be formed by mildly agitating a reaction mixture including an alkyl metal halide and an organic templating agent (e.g., a material used to form the zeolite structure) for a time sufficient to crystallize the reaction mixture and form the zeolite beta (e.g., from about 1 day to many months via hydrothermal digestion), for example. The alkyl metal halide may include silica, alumina, sodium or another alkyl metal oxide, for example. The hydrothermal digestion may occur at temperatures of from slightly below the boiling point of water at atmospheric pressure to about 170° C. at pressures equal to or greater than the vapor pressure of water at the temperature involved, for example.

The cerium promoted zeolite beta may have a silica to alumina molar ratio (expressed as SiO₂/Al₂O₃ ratio) of from about 10 to about 200 or about 50 to 100, for example.

The alkylation catalyst may optionally be bound to, supported on or extruded with any support material. For example, the alkylation catalyst may be bound to a support to increase the catalyst strength and attrition resistance to degradation. The support material may include alumina, silica, aluminosilicate, titanium, silica carbide, and/or clay, for example.

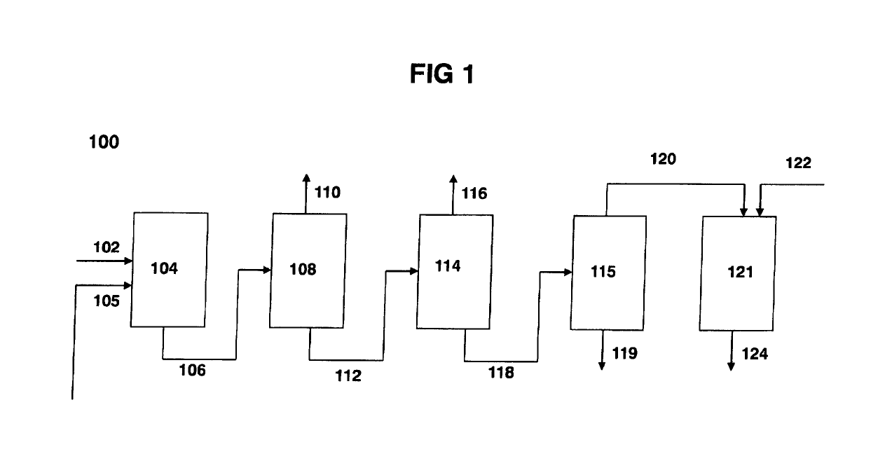

FIG. 1 illustrates a schematic block diagram of an embodiment of liquid-phase alkylation/transalkylation process 100. Process 100 generally includes supplying aromatic input stream 102 to alkylation system 104 (e.g., a first alkylation system.) Aromatic input stream 102 includes at least an aromatic compound. The aromatic compound may include substituted or unsubstituted aromatic compounds. The aromatic compound may include hydrocarbons, such as benzene, for example. If present, the substituents on the aromatic compounds may be independently selected from alkyl, aryl, alkaryl, alkoxy, aryloxy, cycloalkyl, halide and/or other groups that do not interfere with the alkylation reaction, for example.

In one embodiment, the aromatic compound includes one or more hydrocarbons, such as benzene and toluene, for example. In another embodiment, the first aromatic compound includes benzene. The benzene may be supplied from a variety of sources, such as a fresh benzene source and/or a variety of recycle sources, for example. As used herein, the term “fresh benzene source” refers to a source including at least about 95 wt. % benzene, at least about 98 wt. % benzene or at least about 99 wt. % benzene, for example. In one embodiment, the molar ratio of benzene to ethanol may be from about 1:1 to about 30:1, or from about 1:1 to about 20:1, for the total alkylation process including all of the alkylation beds, for example. The molar ratio of benzene to ethanol for individual alkylation beds can range from 5:1 to 100:1, for example.

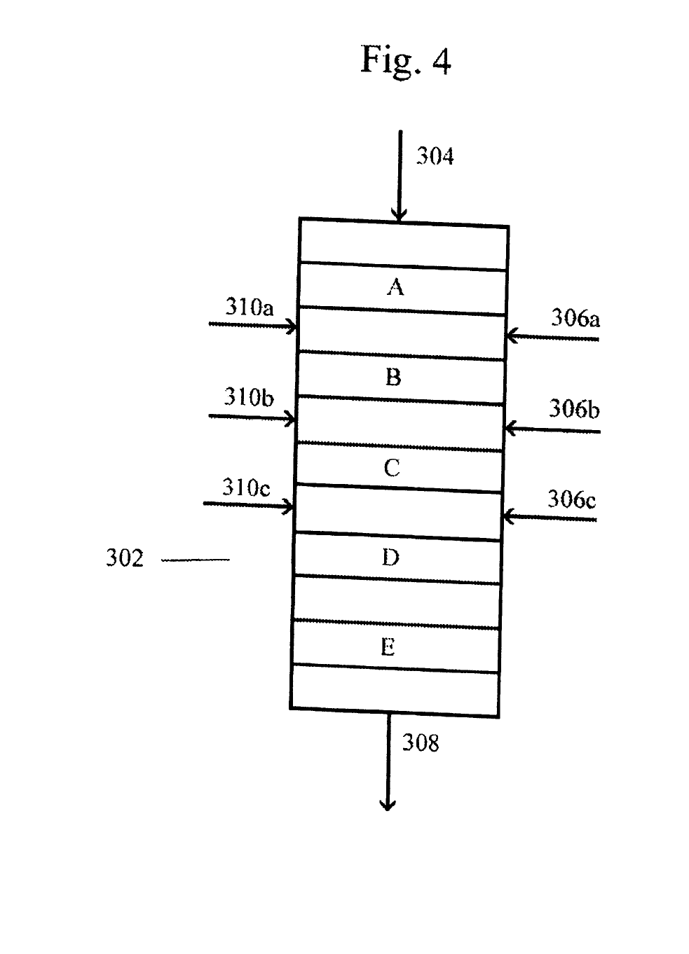

In an alternate embodiment, aromatic input stream 102 further includes ethanol. In another embodiment, ethanol is separately introduced to alkylation system 104 through ethanol input stream 105. In still another embodiment, ethanol may be introduced to alkylation system 104 through both aromatic input stream 102 and ethanol input stream 105. Aromatic input stream 102 and ethanol input stream 105 can be introduced into alkylation system 104 at multiple locations as shown in FIG. 4.

Alkylation system 104 is generally adapted to contact aromatic input stream 102, and, when present, ethanol input stream 105, with an alkylation catalyst to form alkylation output stream 106 (e.g., a first output stream).

At least a portion of alkylation output stream 106 passes to first separation system 108. First overhead fraction line 110 exits first separation system 108 while at least a portion of a first bottoms fraction is passed via first bottoms fraction line 112 to second separation system 114.

A second overhead fraction is generally recovered from second separation system 114 via second overhead fraction line 116 while at least a portion of a second bottoms fraction is passed via second bottoms fraction line 118 to third separation system 115. A third bottoms fraction is generally recovered from third separation system 115 via third bottoms fraction line 119 while at least a portion of a third overhead fraction is passed via third overhead fractions line 120 to transalkylation system 121. In addition to third overhead fraction 120, an additional input, such as additional aromatic compound, such as for instance, benzene, and/or ethanol, is generally supplied to the transalkylation system 121 via transalkylation feed line 122 and contacts the transalkyation catalyst, forming transalkylation output stream 124.

Although not shown herein, the process stream flow may be modified based on unit optimization. For example, at least a portion of any overhead fraction may be recycled as input to any other system within the process. Also, additional process equipment, including but not limited to heat exchangers, filters, water removal systems, and cooling systems may be employed throughout the processes described herein and placement of the process equipment can be as is generally known to one skilled in the art. Further, while described in terms of primary components, the streams indicated may include any additional components as known to one skilled in the art.

The ethanol in input stream 102, transalkylation feed line 122 and, when present, ethanol input stream 105, may contain, in addition to ethanol, a substantial amount of water. In one embodiment of the present disclosure, the ethanol in input stream 102 is at least 25% ethanol, with the remainder being water. In another embodiment of the present disclosure, the ethanol in input stream 102 is about 100% ethanol. In both embodiments, the ethanol may contain minor amounts of other compounds, such as, for instance, aldehydes and ketones.

The alkylation reaction involving ethanol produces water as a byproduct. Further, when the ethanol content in input stream 102, transalkylation feed line 122, and/or ethanol input stream 105 is less than 100% ethanol, a significant amount of water may be present in those respective streams. Water may adversely affect catalyst performance and, under certain circumstances, may deactivate the alkylation or transalkylation catalyst. In some embodiments of the present disclosure, water is removed from the process before, after, or before and after each of the catalyst beds that make up alkylation system 104 and/or transalkylation system 121. In certain embodiments of the present disclosure, where ethanol input stream 105 is less than 100% ethanol, water may be removed after each catalyst bed. In certain other embodiments, where ethanol input stream 105 comprises 100% ethanol, water may be removed, for instance, after every other catalyst bed. Water may be removed by traditional water removal systems. One non-limiting example is a coalescer.

In some embodiments of the present disclosure, the water that is removed from the process 100 may contain ethanol. In certain embodiments, the ethanol-containing water stream may be processed through a stripper to remove at least some of the ethanol from the water. In at least one embodiment where ethanol is stripped from the water, the ethanol may be combined with input stream 102 or ethanol input stream 105.

In addition to the aromatic compound and, where present, the ethanol may further include other compounds in minor amounts (e.g., sometimes referred to as poisons or inactive compounds). Poisons can be nitrogen components such as ammonia, amine compounds, or nitriles, for example. These poisons can be in quantities in the parts-per-billion (ppb) range, but can have significant effect on the catalyst performance and reduce its useful life. In one embodiment, the ethanol and/or benzene includes up to 100 ppb or more of poisons. In one embodiment, the ethanol and/or benzene includes poisons typically ranging from 10 ppb to 50 ppb.

Inactive compounds, which can be referred to as inert compounds, such as C₆ to C₈ aliphatic compounds, may also be present. In one embodiment, the ethanol and/or benzene includes less than about 5% of such compounds or less than about 1%, for example.

Alkylation system 104 can include a plurality of multi-stage reaction vessels. In one embodiment, the multi-stage reaction vessels can include a plurality of operably connected catalyst beds, such beds containing an alkylation catalyst, such as shown in FIG. 4 for example. Such reaction vessels are generally liquid phase reactors operated at reactor temperatures and pressures sufficient to maintain the alkylation reaction in the liquid phase, i.e., the aromatic compound is in the liquid phase. Such temperatures and pressures are generally determined by individual process parameters. For example, the reaction vessel temperature may be from 65° C. to 350° C. or from 200° C. to 300° C. The reaction vessel pressure may be any suitable pressure in which the alkylation reaction can take place in the liquid phase, such as from 300 psig to 1,200 psig, for example.

In one embodiment, the space velocity of the reaction vessel within alkylation system 104 is from 1.0 liquid hourly space velocity (LHSV) per bed to 100 LHSV per bed, based on the aromatic feed rate. In alternate embodiments, the LHSV can range from 2 to 100, or from 4 to 50. For the alkylation process overall, including all of the alkylation beds of the preliminary alkylation reactor or reactors and the primary alkylation reactor or reactors, the space velocity can range from 1 LHSV to 50 LHSV.

Akylation output stream 106 generally includes a second aromatic compound. In one embodiment, the second aromatic compound includes ethylbenzene, for example.

First separation system 108 may include any process or combination of processes known to one skilled in the art for the separation of aromatic compounds. For example, first separation system 108 may include one or more distillation columns (not shown,) either in series or in parallel. The number of such columns may depend on the volume of alkylation output stream 106 passing through.

First overhead fraction line 110 from first separation system 108 generally includes the first aromatic compound, such as benzene, for example.

First bottoms fraction line 112 from the first separation system 108 generally includes the second aromatic compound, such as ethylbenzene, for example.

Second separation system 114 may include any process known to one skilled in the art, for example, one or more distillation columns (not shown), either in series or in parallel.

Second overhead fraction line 116 from second separation system 114 generally includes the second aromatic compound, such as ethylbenzene, which may be recovered and used for any suitable purpose, such as the production of styrene, for example. Production of styrene from ethylbenzene may be performed by traditional processes including, but not limited to, catalytic dehydrogenation.

Second bottoms fraction line 118 from second separation system 114 generally includes heavier aromatic compounds, such as polyethylbenzene, cumene and/or butylbenzene, for example.

Third separation system 115 generally includes any process known to one skilled in the art, for example, one or more distillation columns (not shown), either in series or in parallel.

In a specific embodiment, third overhead fraction line 120 from third separation system 115 may include diethylbenzene and triethylbenzene, for example. Third bottoms fraction line 119 (e.g., heavies) may be recovered from third separation system 115 for further processing and recovery (not shown).

Transalkylation system 121 generally includes one or more reaction vessels having a transalkylation catalyst disposed therein. The reaction vessels may include any reaction vessel, combination of reaction vessels and/or number of reaction vessels (either in parallel or in series) known to one skilled in the art.

The transalkylation catalyst may include a large-pore catalyst and may be the same catalyst or a different catalyst than the alkylation catalyst, for example. In one embodiment, the transalkylation catalyst comprises at least one large pore catalyst. Suitable large pore catalysts include zeolite beta, zeolite Y, zeolite MCM-22, zeolite MCM-36, zeolite MCM-49 or zeolite MCM-56, for example. In one specific embodiment, the alkylation catalyst is Zeolyst CP 787 H-Beta Extrudate, available from Zeolyst International.

Transalkylation output stream 124 generally includes the second aromatic compound, for example, ethylbenzene. Transalkylation output stream 124 can be sent to one of the separation systems, such as first separation system 108, for separation of the components of transalkylation output stream 124.

In one embodiment, transalkylation system 121 is operated under liquid phase conditions. For example, transalkylation system 121 may be operated at a temperature of from about 65° C. to about 290° C. and a pressure of about 800 psig or less.

In a specific embodiment, benzene is recovered through first overhead fraction line 110 and recycled (not shown) as input to alkylation system 104, while ethylbenzene and/or polyalkylated benzenes are recovered via first bottoms fraction line 112.

As previously discussed, alkylation system 104 generally includes an alkylation catalyst. Aromatics input stream 102, e.g., benzene/ethanol, contacts the alkylation catalyst during the alkylation reaction to form alkylation output 106, e.g., ethylbenzene.

In an unexpected development, the process described herein resulted in a near 100% incorporation of ethanol.

Unfortunately, alkylation catalyst systems generally experience deactivation requiring either regeneration or replacement. Additionally, alkylation processes generally require periodic maintenance. Both circumstances generally produce disruptions for liquid phase alkylation processes. The deactivation results from a number of factors. One of those factors is that poisons present in the aromatics input stream 102, such as nitrogen, sulfur and/or oxygen containing impurities, either naturally occurring or a result of a prior process, may reduce the activity of the alkylation catalyst.

Embodiments of the disclosure provide a process wherein continuous production during catalyst regeneration and maintenance may be achieved. For example, one reactor may be taken off-line for regeneration of the catalyst, either by in-situ or ex-situ methods, while the remaining reactor may remain on-line for production. The determination of when such regeneration will be required can depend on specific system conditions, but is generally a predetermined set point (e.g., catalyst productivity, temperature, or time).

If in-situ regeneration is not possible, when removing the catalyst from the reactor for regeneration, it may be possible to replace the catalyst and place the reactor on-line while the removed/deactivated catalyst is regenerated. In such an embodiment, the cost of replacing the catalyst can be large and therefore it is beneficial that such catalyst should have a long life before regeneration. Embodiments of the disclosure may provide an alkylation system capable of extended catalyst life and extended production runs.

In certain embodiments of the present disclosure, aromatics input stream 102 may be treated to remove these poisons prior to being fed to alkylation reactor 104. In some embodiments where poison removal is accomplished prior to alkylation reactor 104, a swing reactor configuration is used, as described in U.S. application Ser. No. 13/028,381, Use of Swing Preliminary Alkylation Reactors, filed Feb. 16, 2011, which is fully incorporated herein by reference.

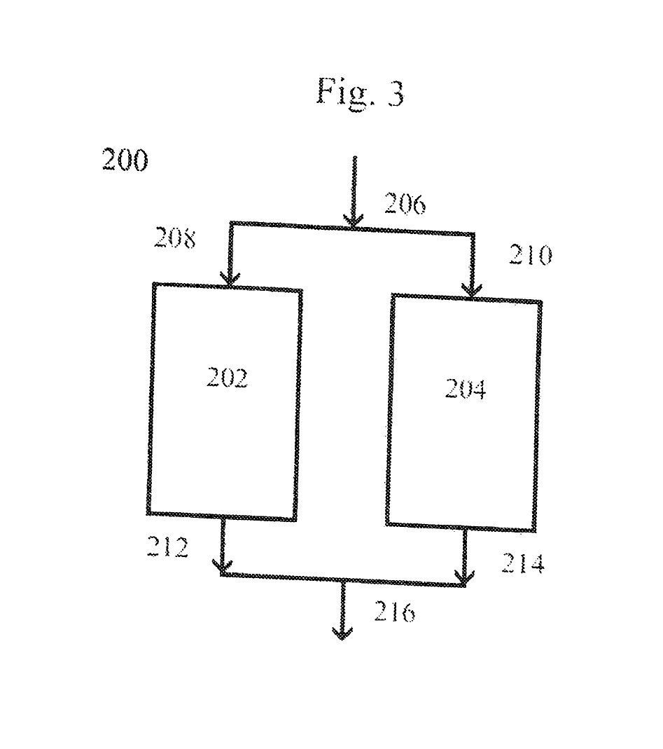

FIG. 3 illustrates a non-limiting embodiment of an alkylation system 200, which can be a preliminary alkylation system. The alkylation system 200 shown includes a plurality of alkylation reactors, such as two alkylation reactors 202 and 204, operating in parallel. One or both alkylation reactors 202 and 204, which may be the same type of reaction vessel, or, in certain embodiments, may be different types of reaction vessels, may be placed in service at the same time. For example, only one alkylation reactor may be on line while the other is undergoing maintenance, such as catalyst regeneration. In one embodiment, the alkylation system 200 is configured so that the input stream is split to supply approximately the same input to each alkylation reactor 202 and 204. However, such flow rates will be determined by each individual system.

This mode of operation (e.g., multiple parallel reactors) may involve operation of the individual reactors at relatively lower space velocities for prolonged periods of time with periodic relatively short periods of operation at enhanced, relatively higher space velocities when one reactor is taken off-stream. By way of example, during normal operation of the system 200, with both reactors 202 and 204 on-line, the input 206 stream may be supplied to each reactor (e.g., via lines 208 and 210) to provide a reduced space velocity. The output 216 stream may be the combined flow from each reactor (e.g., via lines 212 and 214). When a reactor is taken off-line and the feed rate continues unabated, the space velocity for the remaining reactor may approximately double.

In a specific embodiment, one or more of the plurality of alkylation reactors may include a plurality of interconnected catalyst beds. The plurality of catalyst beds may include from 2 to 15 beds, or from 5 to 10 beds or, in specific embodiments, 5 or 8 beds, for example. Embodiments can include one or more catalyst beds having a mixed catalyst load that includes a medium pore molecular sieve catalyst and one or more other catalysts. The mixed catalyst load can, for example, be a layering of the various catalysts, either with or without a barrier or separation between them, or alternately can include a physical mixing where the various catalysts are in contact with each other.

FIG. 4 illustrates a non-limiting embodiment of an alkylation reactor 302 for use in liquid phase alkylation. The alkylation reactor 302 includes five series connected catalyst beds designated as beds A, B, C, D, and E. In one embodiment, an input stream 304 (e.g., benzene/ethanol or benzene) is introduced to the reactor 302, passing through each of the catalyst beds to contact the alkylation catalyst and form the alkylation output 308. Additional alkylating agent (i.e. ethanol) may be supplied via lines 306a, 306b, and 306c to the interstage locations in the reactor 302. Additional aromatic compound may also be introduced to the interstage locations via lines 310a, 310b and 310c, for example.

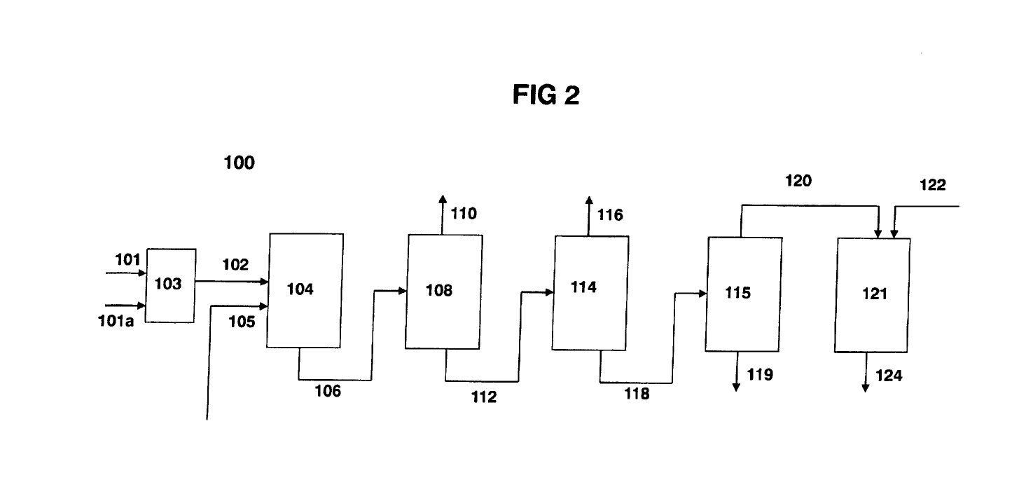

Referring to FIG. 2, in certain embodiments, alkylation/transalkylation system 100 may further include a preliminary alkylation system 103. Preliminary alkylation system 103 may be maintained at ambient or up to alkylation conditions, for example.

Preliminary alkylation input stream 101 may be passed through preliminary alkylation system 103 prior to entry into alkylation system 104 to reduce the level of poisons in aromatics input stream 102, for example. In one embodiment, the level of poisons is reduced by at least 10%, or at least 25% or at least 40% or at least 60% or at least 80%, for example. For example, preliminary alkylation system 103 may be used as a sacrificial system, thereby reducing the amount of poisons contacting the alkylation catalyst in alkylation system 104 and reducing the frequency of regeneration needed of the alkylation catalyst in alkylation system 104.

Preliminary alkylation system 103 generally includes a preliminary alkylation catalyst disposed therein. The alkylation catalyst, transalkylation catalyst and/or the preliminary alkylation catalyst may be the same or different.

As a result of the level of poisons present in preliminary alkylation input 101, the preliminary catalyst in the preliminary alkylation system 103 has typically deactivated rapidly, requiring frequent regeneration and/or replacement. For example, the preliminary catalyst may experience deactivation more rapidly than the alkylation catalyst (e.g., from about twice as often to about 1.5 times as often). Previous systems have generally used the preliminary alkylation system 103 as a sacrificial system, thereby reducing the amount of poisons contacting the alkylation catalyst in alkylation system 104.

However, embodiments of the invention utilize a catalyst having a lower SiO₂/Al₂O₃ ratio than those preliminary alkylation catalysts previously used (and discussed herein). For example, the preliminary alkylation catalyst may have a SiO₂/Al₂O₃ ratio that is about 50 or less, or that is about 25 or less, or that is from about 5 to about 50 or from about 7.5 to about 25, for example.

In one specific, non-limiting embodiment, the preliminary alkylation catalyst has a SiO₂/Al₂O₃ ratio that is lower than the SiO₂/Al₂O₃ ratio of the alkylation catalyst. For example, the preliminary alkylation catalyst may have a SiO₂/Al₂O₃ ratio that is at least about 25%, or at least about 50%, or at least about 75% or at least about 90% lower than the SiO₂/Al₂O₃ ratio of the alkylation catalyst.

The preliminary alkylation catalyst may include any commercially available catalyst having the SiO₂/Al₂O₃ ratio discussed herein. For example, the preliminary alkylation catalyst may include Y-84 zeolite (i.e., SiO₂/Al₂O₃ ratio of 9.1), for example.

Further, while not described in detail herein, it is contemplated that the preliminary alkylation catalyst may include a plurality of preliminary alkylation catalysts so long as at least one of the plurality of preliminary alkylation catalysts include the lower SiO₂/Al₂O₃ ratio preliminary alkylation catalyst described herein.

The SiO₂/Al₂O₃ ratio is inversely proportional to the number of acid sites per unit mass of the catalyst. Therefore, if a first catalyst has to a higher SiO₂/Al₂O₃ ratio than a second catalyst, the first catalyst has a lower number of acid sites than the second catalyst. Thus the present process employs a catalyst in preliminary alkylation system 103 that has a greater number of acid sites per unit mass than the catalyst in alkylation system 104. Apart from the difference in the number of acid sites per unit mass of the catalyst, the first and second alkylation catalysts may be the same or different.

In one embodiment, the ratio of the number of acid sites per unit mass of the catalyst in preliminary alkylation system 103 to the number of acid sites per unit mass of the catalyst in alkylation system 104 is in the range of 40:1 to 1:1, and generally in the range of 10:1 to 1:1. The number of acid sites per unit mass of a catalyst can be determined by variety of techniques including, but not limited to, Bronsted proton measurement, tetrahedral aluminum measurement, the adsorption of ammonia, pyridine and other amines, and the rate constant for the cracking of hexane.

In one embodiment the preliminary alkylation input stream 101 comprises the entire benzene feed to the process and a portion of the ethanol feed to the process. In another embodiment, the portion of the ethanol feed to the process enters the preliminary alkylation system 103 through preliminary ethanol feed stream 101a. The feed streams(s) pass(es) through preliminary alkylation system 103 that contains zeolite catalyst prior to entry into the alkylation system 104 to reduce the level of poisons contacting the alkylation catalyst in the alkylation system 104. The aromatic input stream 102 from the preliminary alkylation system 103 can include unreacted benzene and ethylbenzene produced from preliminary alkylation system 103. Additional ethanol can be added to the alkylation system 104 through ethanol feed stream 105 to react with the unreacted benzene. In this embodiment the preliminary alkylation system 103 can reduce the level of poisons in the benzene and that portion of the ethanol feed that is added to the process preliminary alkylation input stream 101. Ethanol that is added after the preliminary alkylation system 103, such as to the alkylation system 104 through ethanol feed stream 105, would not have a reduction in the level of poisons from the preliminary alkylation system 103.

As a result of the level of poisons present in the preliminary alkylation input 101, the preliminary catalyst in the preliminary alkylation system 103 may become deactivated, requiring regeneration and/or replacement. For example, the preliminary catalyst may experience deactivation more rapidly than the alkylation catalyst.

When regeneration of any catalyst within the system is desired, the regeneration procedure generally includes processing the deactivated catalyst at high temperatures, although the regeneration may include any regeneration procedure known to one skilled in the art.

Once a reactor is taken off-line, the catalyst disposed therein may be purged. Off-stream reactor purging may be performed by contacting the catalyst in the off-line reactor with a purging stream, which may include any suitable inert gas (e.g., nitrogen), for example. The off-stream reactor purging conditions are generally determined by individual process parameters and are generally known to one skilled in the art.

The catalyst may then undergo regeneration. The regeneration conditions may be any conditions that are effective for at least partially reactivating the catalyst and are generally known to one skilled in the art. For example, regeneration may include heating the alkylation catalyst to a temperature or a series of temperatures, such as a regeneration temperature of from about 200° C. to about 500° C. above the purging or alkylation reaction temperature, for example.

In one embodiment, the alkylation catalyst is heated to a first temperature (e.g., 400° C.) with a gas containing nitrogen and 2 mol % or less oxygen, for example, for a time sufficient to provide an output stream having an oxygen content of about 0.1 mol %. The regeneration conditions will generally be controlled by the alkylation system restrictions and/or operating permit requirements that can regulate conditions, such as the permissible oxygen content that can be sent to flare for emission controls. The alkylation catalyst may then be heated to a second temperature (e.g., 500° C.) for a time sufficient to provide an output stream having a certain oxygen content. The catalyst may further be held at the second temperature for a period of time, or at a third temperature that is greater than the second temperature, for example. Upon catalyst regeneration, the reactor is allowed to cool and can then be made ready to be placed on-line for continued production.

In certain other embodiments of the invention, a molecular sieve catalyst is used in a gas phase alkylation process. In one embodiment, the alkylation catalyst employed in the alkylation zone(s) or the alkylation catalyst employed in each alkylation reaction zone, and transalkylation zone, including the reactive guard bed as described below, comprises at least one medium pore molecular sieve having, for example, a Constraint Index of 2-12 (as defined in U.S. Pat. No. 4,016,218). Suitable medium pore molecular sieves include ZSM-5, ZSM-11, ZSM-12, ZSM-22, ZSM-23, ZSM-35, and ZSM-48. ZSM-5 is described in detail in U.S. Pat. Nos. 3,702,886 and Re. 29,948. ZSM-11 is described in detail in U.S. Pat. No. 3,709,979. ZSM-12 is described in U.S. Pat. No. 3,832,449. ZSM-22 is described in U.S. Pat. No. 4,556,477. ZSM-23 is described in U.S. Pat. No. 4,076,842. ZSM-35 is described in U.S. Pat. No. 4,016,245. ZSM-48 is more particularly described in U.S. Pat. No. 4,234,231. The catalyst used in any zone may be the same or different as that used in any other zone.

The alkylation system of FIG. 1 described above with respect to liquid phase alkylation is applicable to gas phase as described above. Further, water removal may be performed for gas phase alkylation because of the sensitivity of gas phase catalysts to water.

### EXAMPLES

### Example 1

### Liquid Phase

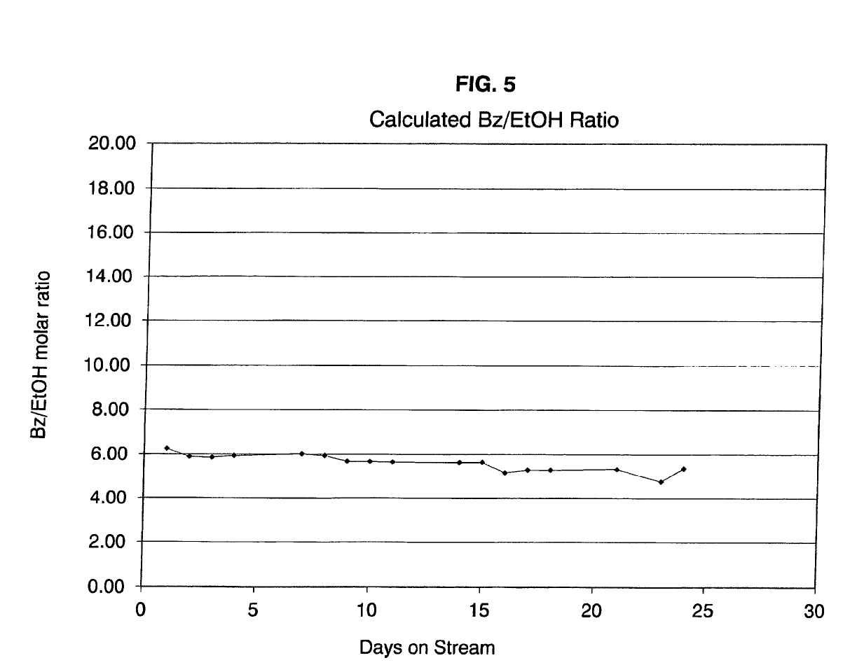

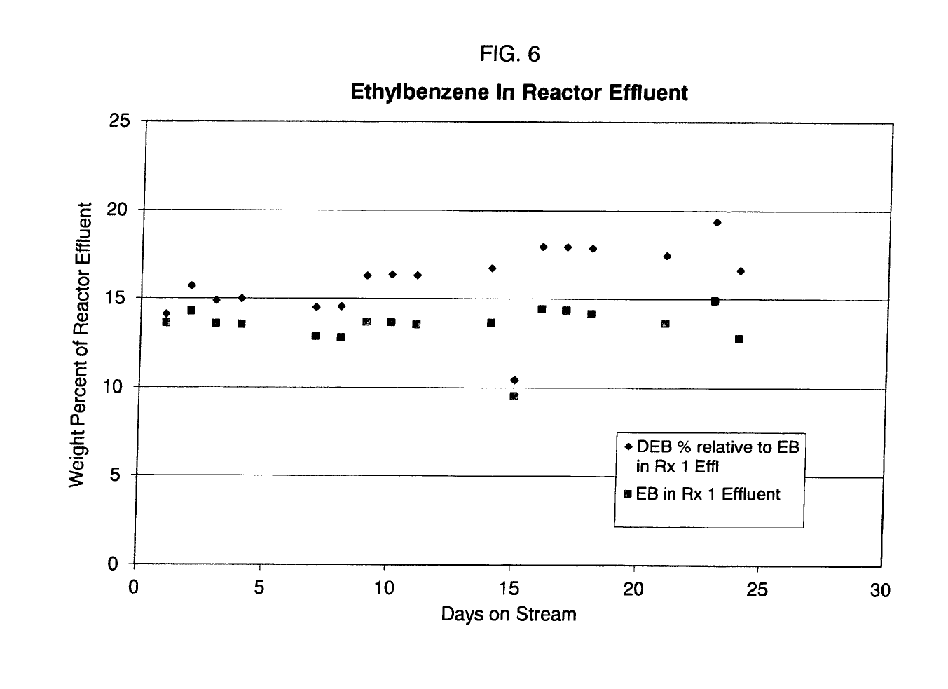

Liquid phase alkylation was tested over a period of 25 days using as feed fresh benzene and 95% pure ethanol. The reactor bed was charged with 14.35 grams of ZHB-4 catalyst. The benzene:ethanol molar feed ratio versus days on stream is shown in FIG. 5. Ethylbenzene content in the reactor effluent versus days on stream is shown in FIG. 6. Benzene to ethylbenzene conversion was as high as 16% in the 30 day run. Diethylbenzene percent relative to ethylbenzene is also shown in FIG. 6.

### Example 2

### Gas Phase

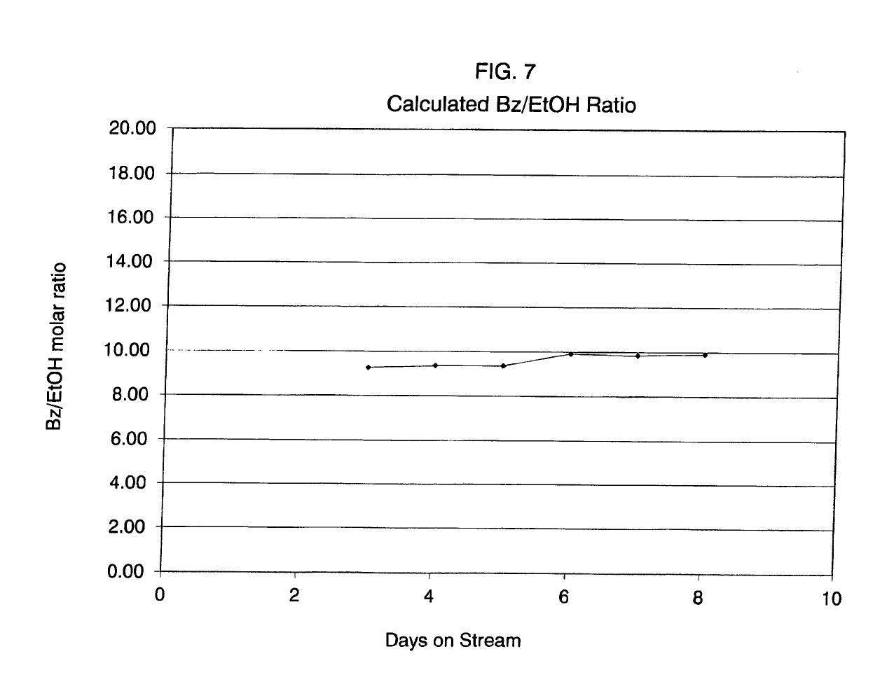

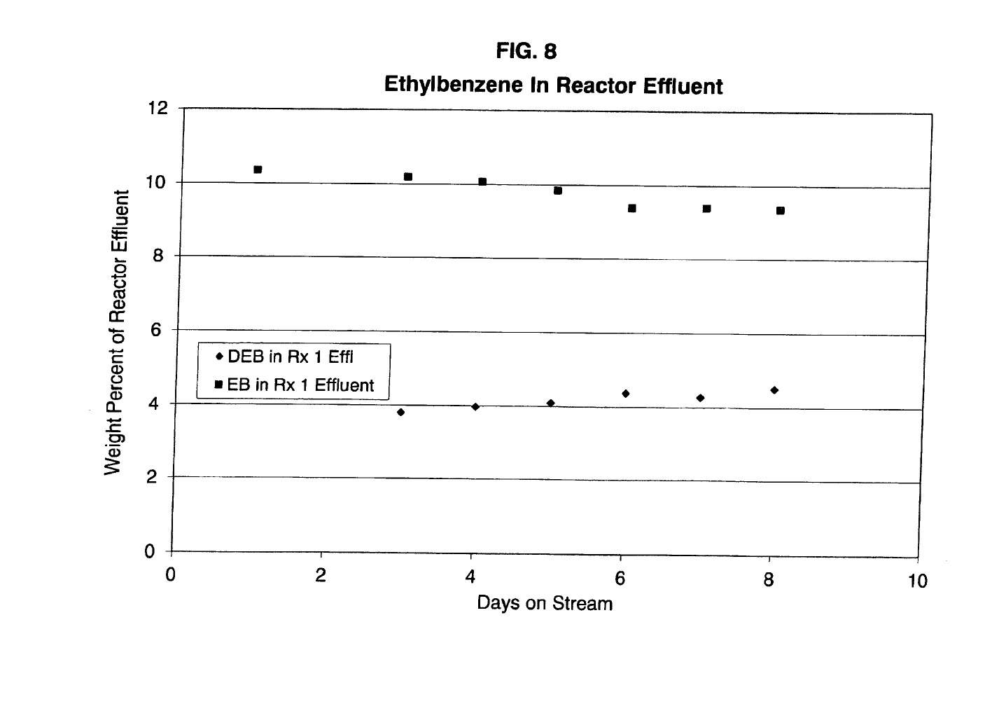

Gas phase alkylation was tested over a period of 8 days using as feed fresh benzene and 95% pure ethanol. 5.81 grams of EBUF-1 catalyst was used in the gas phase reactor. The benzene:ethanol molar feed ratio versus days on stream is shown in FIG. 7. Ethylbenzene and diethylbenzene content in the reactor effluent versus days on stream is shown in FIG. 8. Ethylbenzene in the reactor effluent initially exceeded 10% by weight.

While the foregoing is directed to embodiments of the present invention, other and further embodiments of the invention may be devised without departing from the basic scope thereof and the scope thereof is determined by the claims that follow.

What is claimed is:

1. A method of producing an alkylaromatic comprising contacting an aromatic with ethanol in the presence of a catalyst at liquid phase alkylation conditions to form the alkylaromatic.

2. The method of claim 1, wherein alkylaromatic is ethyl benzene and the aromatic is benzene.

3. The method of claim 2, wherein ethanol contains no more than 75% water.

4. The method of claim 4, wherein the ethanol is 100% ethanol.

5. The method of claim 1, wherein the catalyst is selected from the group consisting of zeolite beta, zeolite Y, zeolite MCM-22, zeolite MCM-36, zeolite MCM-49 and zeolite MCM-56.

6. The method of claim 5, wherein the catalyst is a zeolite beta and the zeolite beta has a sodium content of less than about 0.2 wt %.

7. The method of claim 5, wherein the catalyst is modified with a rare earth selected from the group consisting of cerium, lanthanum, praseodymium and ytterbium.

8. A method of producing an alkylaromatic, the method comprising:
providing at least one reaction zone containing a zeolite catalyst;
introducing a feed stream comprising an aromatic and ethanol to the reaction zone; and
reacting at least a portion of the aromatic under alkylation conditions to produce an alkylaromatic.

9. The method of claim 8, wherein the at least one reaction zone comprises:
a preliminary alkylation system containing a preliminary alkylation catalyst so as to alkylate the aromatic compound and form a preliminary output stream; and
a primary alkylation system adapted to receive the preliminary output stream and contact the preliminary output stream and an alkylating agent with a primary alkylation catalyst disposed therein so as to form a primary outlet stream.

10. The method of claim 9, wherein the feed stream further comprises catalyst poisons averaging at least 5 ppb.

11. The method of claim 8, wherein the aromatic is benzene.

12. The method of claim 8 further comprising a plurality of reaction zones, wherein the reaction zones are connected in series.

13. The method of claim 12 further comprising after the reacting step:
removing a water stream from between the reaction zones.

14. The method of claim 8 further comprising:
providing a separation system, wherein the separation system is fluidly connected to the at least one reaction zone; and
separating the alkylaromatic from the aromatic.

15. The method of claim 8, wherein the catalyst in the first preliminary alkylation reactor can be regenerated in-situ.

16. The method of claim 8, wherein the first preliminary alkylation reactor can be bypassed for catalyst regeneration without taking the at least one primary alkylation reactor out of service.

17. The method of claim 16, wherein the primary alkylation reactor experiences a decrease in catalyst deactivation when the preliminary alkylation reactor is in service.

18. A process for producing an alkylaromatic compound, the process comprising:
(a) introducing an input stream comprising an aromatic hydrocarbon, and an alkylating agent comprising ethanol into a preliminary alkylation system comprising a preliminary alkylation catalyst having a first SiO₂/Al₂O₃ ratio, said preliminary alkylation catalyst is a zeolite;
(b) operating said preliminary alkylation system under alkylation conditions to produce said alkylaromatic compound;
(c) withdrawing from said preliminary alkylation system a first output stream comprising said alkylaromatic compound and unreacted aromatic hydrocarbon;
(d) introducing at least part of said first output stream and ethanol into a first alkylation system comprising a first alkylation catalyst having a second SiO₂/Al₂O₃ ratio, said first alkylation catalyst is a molecular sieve, wherein the preliminary alkylation catalyst and the first alkylation catalyst are different in that the preliminary alkylation catalyst has a lower SiO₂/Al₂O₃ ratio than the first alkylation catalyst whereby the frequency at which any alkylation catalyst is removed for replacement, regeneration or reactivation is reduced as compared to either alkylation catalyst alone;
(e) operating said first alkylation system under alkylation conditions to produce said alkylaromatic compound; and
(f) withdrawing from said first alkylation system a second output stream comprising said alkylaromatic compound.

19. The process of claim 18, wherein the preliminary alkylation catalyst has a first amount of acid sites per unit mass of the preliminary alkylation catalyst and the first alkylation catalyst has a second amount of acid sites per unit mass of the first alkylation catalyst and wherein the preliminary alkylation catalyst has a greater number of acid sites per unit mass than the first alkylation catalyst.

20. A process of producing ethylbenzene by the alkylation of benzene with ethanol, the process comprising:
providing at least one reaction zone comprising a zeolite catalyst;
introducing a feed stream comprising benzene and ethanol to the reaction zone; and
reacting at least a portion of the benzene with ethanol under alkylation conditions to produce ethylbenzene.

21. A process of producing styrene comprising catalytically dehydrogenating the ethyl benzene of claim 20 to form styrene.
